(19) United States
(12) Patent Application Publication
(10) Pub. No.: US 2021/0093899 A1
(43) Pub. Date: Apr. 1, 2021

(54) NEGATIVE ION AIR-PERMEABLE MASK

(71) Applicant: YU-FEN JUSTINE HUANG, Taipei (TW)

(72) Inventor: YU-FEN JUSTINE HUANG, Taipei (TW)

(21) Appl. No.: 16/586,716

(22) Filed: Sep. 27, 2019

(51) Int. Cl. A62B 7/10; A62B 23/02; A61L 9/14

(52) U.S. Cl. CPC A62B 7/10; A61L 9/14; A62B 23/025

(57) ABSTRACT

A negative ion air-permeable mask comprises an outer cover layer, a filter layer and an insulating layer, the filter layer is disposed on one side of the outer cover layer, and the insulating layer is disposed at a position of another side of the filter layer opposite to the outer cover layer. The insulating layer is provided with a negative ion optimization module having a power supply component, a negative ion generator and a negative ion kinetic-energy optimizer. Thereby, the negative ion air-permeable mask can repeatedly utilize a fullerene material to perform corona discharge through the negative ion optimization module to generate a maximum quantified small molecular negative ions to provide fresh air, combine and eliminate suspended particles, and simultaneously circulate the air through a vent between the outer cover layer, the filter layer and the insulating layer.

### BACKGROUND OF THE INVENTION

### Field of Invention

This present invention relates to a negative ion air-permeable mask capable of producing negative ions, having a venting effect, and capable of replacing a filter mesh.

### Related Art

In recent years, outbreaks of various epidemic and infectious diseases have been reported, which means that there are many bacteria lurking around us, and some are infected through contact or through droplets. Although the infection routes are different, they will cause different degrees of harm to the human body. Due to the rapid development of industry and commerce, the quantity of suspended particles and harmful substances in the environment is greatly increased, and the suspended particles (such as PM2.5) floating in the air will cause great harm to the human body; according to published articles by scholars, it is a potential factor in inducing cancer. Therefore, people wear masks when they are out to avoid inhaling excessive suspended particles or harmful substances. However, the masks currently sold on the market are mainly categorized into disposable type and medical-grade complete enclosure type. The disposable masks are mostly thin with one or two layers, which can only be used to block the infection of droplets, but they are not effective for blocking tiny bacteria or suspended particles (such as PM2.5). Even though medical grade masks have functional materials for completely isolation, but they are completely airtight and blocking breathing, so further research and improvement are still necessary for the masks on the market.

Therefore, how to improve the above-mentioned drawbacks is the technical difficulty that the inventor of the present invention wants to solve.

### SUMMARY OF THE INVENTION

Therefore, in order to effectively solve the above problems, a main object of the present invention is to provide a negative ion air-permeable mask capable of generating negative ions and having an air circulation effect, and capable of conveniently replacing a filter mesh.

In order to achieve the above object, the present invention provides a negative ion air-permeable mask comprising an outer cover layer, a filter layer and an insulating layer, wherein the outer cover layer has a first side and a second side opposite to a position of the first side, the filter layer is disposed at a position of the second side of the outer cover layer, and the insulating layer is disposed at a position of another side of the filter layer opposite to the outer cover layer. The insulating layer is provided with a negative ion optimization module. The negative ion optimization module has a power supply component, a negative ion generator and a negative ion kinetic-energy optimizer. Thereby, the negative ion air-permeable mask can generate small molecular negative ions through the negative ion optimization module to provide users with fresh air, adsorb and settle suspended particles, and simultaneously circulate the air through a vent between the outer cover layer, the filter layer and the insulating layer, so that the negative ion air-permeable mask is capable of achieving the efficacies of generating negative ions and providing a ventilating effect.

According to one embodiment of the negative ion air-permeable mask of the present invention, wherein the filter layer has an air inlet side and an air outlet side, and an air inlet is formed and an air intake checker is provided between the air inlet side and a bottom of the second side of the outer cover layer.

According to one embodiment of the negative ion air-permeable mask of the present invention, wherein an air outlet is formed and an air outlet checker is provided between a joint side of the insulating layer and a top of the air outlet side.

According to one embodiment of the negative ion air-permeable mask of the present invention, wherein a power supply component, a negative ion generator and a negative ion kinetic-energy optimizer are disposed in the negative ion optimization module.

According to one embodiment of the negative ion air-permeable mask of the present invention, wherein the negative ion optimization module is further disposed with a charging connector, a switch, a negative ion outlet, an outer insulating resin plate and an inner conductive metal plate.

According to one embodiment of the negative ion air-permeable mask of the present invention, wherein the negative ion kinetic-energy optimizer has an insulative housing and a metal vacuum cylinder, and inside the metal vacuum cylinder has a plurality of metal powders.

According to one embodiment of the negative ion air-permeable mask of the present invention, wherein the charging connector is electrically connected to the power supply component, the power supply component is electrically connected to the negative ion generator by at least one first power supply line, electrically connected to the negative ion kinetic-energy optimizer by at least one second power supply line, and electrically connected to the inner conductive metal plate by at least one third power supply line. The negative ion generator produces a large amount of negative ions by corona discharge using a carbon material of needle-like fullerene honeycomb structure, and is electrically connected to the negative ion kinetic-energy optimizer via a conductive negative ion output wire, and the negative ions are sent into the negative ion kinetic-energy optimizer to receive impact of the metal powders with electrodes and absorb a large amount of kinetic energy. The negative ion kinetic-energy optimizer further has a conductive high-kinetic energy negative ion output wire electrically connected to the inner conductive metal plate. Once again, a fullerene release tip linked to the conductive metal plate releases small molecular negative ions to the human body within a short distance.

According to one embodiment of the negative ion air-permeable mask of the present invention, wherein the insulating layer has a skin fitted portion.

According to one embodiment of the negative ion air-permeable mask of the present invention, wherein each side of the outer cover layer has an ear hanger.

According to one embodiment of the negative ion air-permeable mask of the present invention, wherein each side of the outer cover layer has an earmuff, and a head strip is formed between the two earmuffs.

### BRIEF DESCRIPTION OF THE DRAWINGS

FIG. 1 is a perspective assembly view of a preferred embodiment of a negative ion air-permeable mask of the present invention;

FIG. 2 is a perspective exploded view of a preferred embodiment of the negative ion air-permeable mask of the present invention;

FIG. 3 is a cross-sectional view of a preferred embodiment of the negative ion air-permeable mask of the present invention;

FIG. 4 is a perspective view of a negative ion optimization module of the present invention;

FIG. 5 is a partial perspective view of the negative ion optimization module of the present invention;

FIG. 6 is a first schematic view of the implementation of a preferred embodiment of the negative ion air-permeable mask of the present invention;

FIG. 7 is a second schematic view of the implementation of a preferred embodiment of the negative ion air-permeable mask of the present invention; and

FIG. 8 is a third schematic view of the implementation of a preferred embodiment of the negative ion air-permeable mask of the present invention.

### DETAILED DESCRIPTION OF THE INVENTION

The above objects of the present invention, as well as its structural and functional features, will be described in accordance with the preferred embodiments of the drawings.

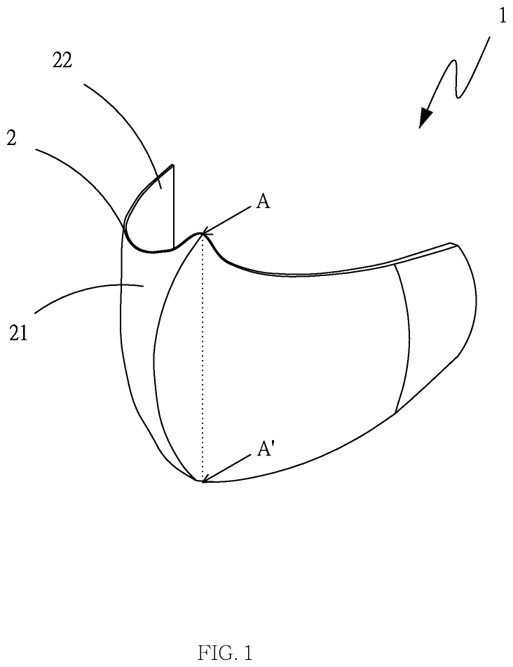

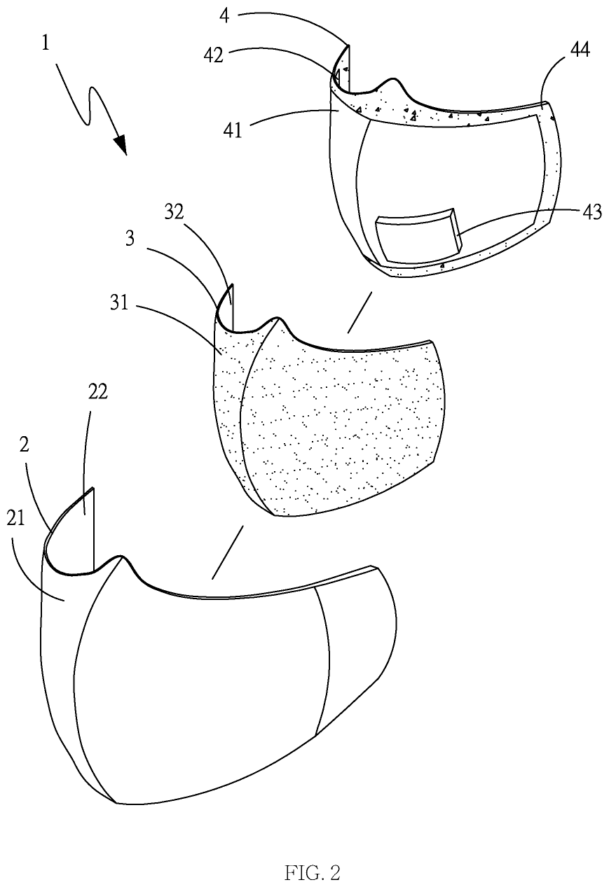

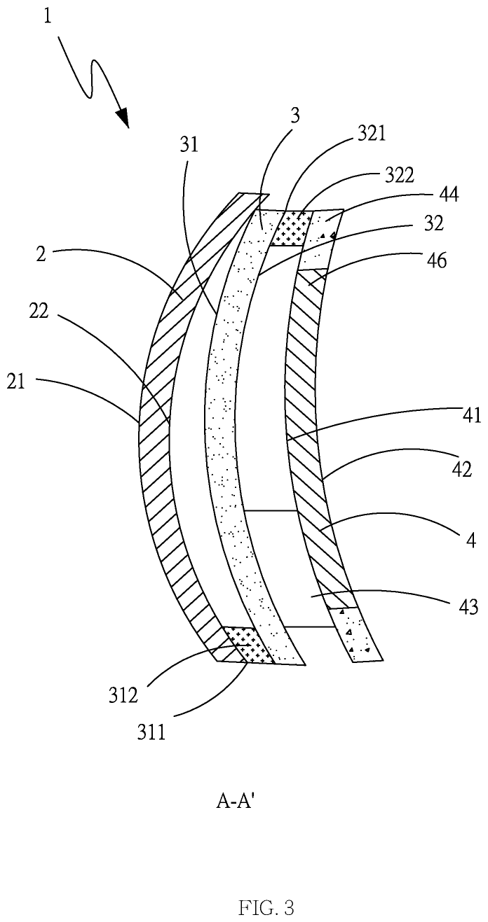

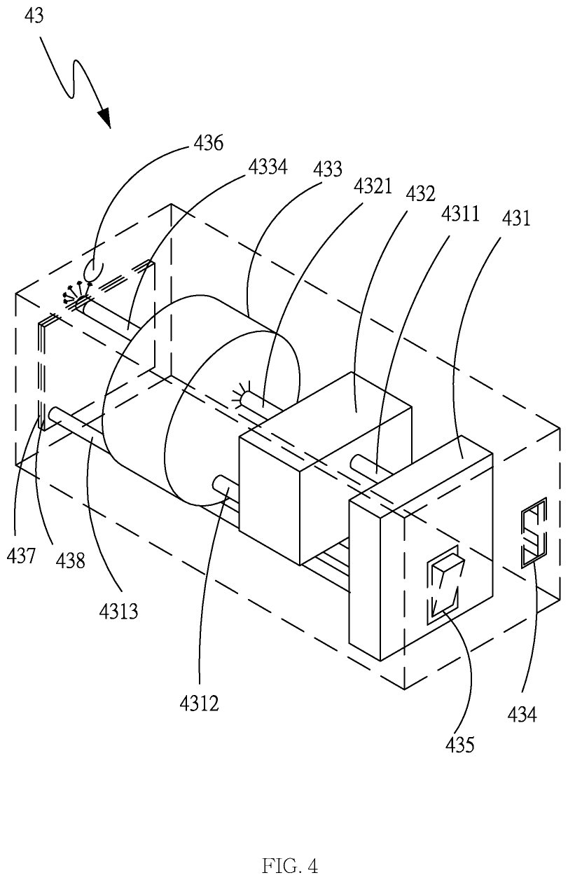

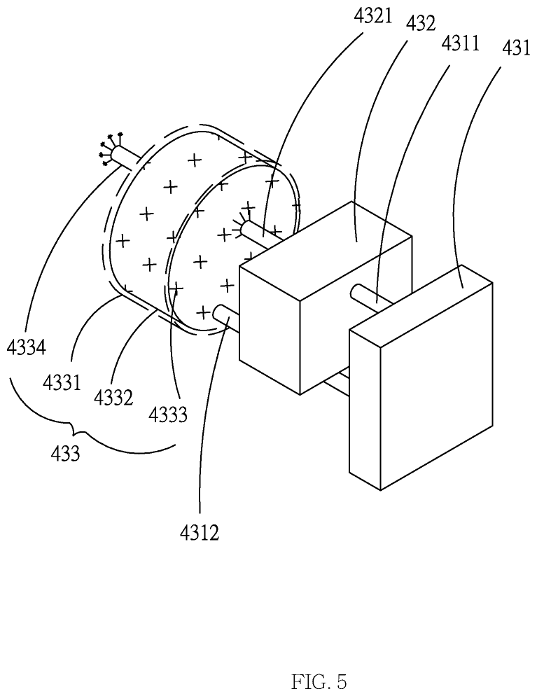

Please refer to FIG. 1 to FIG. 5, which are respectively perspective assembly view, perspective exploded view and cross-sectional view of a preferred embodiment of a negative ion air-permeable mask of the present invention, as well perspective view and partial perspective view of a negative ion optimization module of the present invention. It is clearly shown in the drawings that a negative ion air-permeable mask 1 comprises an outer cover layer 2, a filter layer 3 and an insulating layer 4.

Wherein the outer cover layer 2 has a first side 21 formed at a position of one side, and a second side 22 formed at another side opposite to the first side 21.

In addition, the filter layer 3 is disposed at a position of the second side 22 of the outer cover layer 2, the filter layer 3 is formed with an air inlet side 31 at a side opposite to the second side 22, and the filter layer 3 is formed with an air outlet side 32 at another side opposite to the air inlet side 31. The filter layer 3 is partially assembled on the second side 22 with the air inlet side 31, and an air inlet 311 is formed and an air intake checker 312 is provided between the air inlet side 31 and a bottom of the second side 22.

In addition, the insulating layer 4 is disposed at a position of the air outlet side 32 of the filter layer 3, the insulating layer 4 is formed with a joint side 41 opposite to the air outlet side 32, and the insulating layer 4 is formed with a contact side 42 at another side opposite to the joint side 41. A negative ion optimization module 43 is disposed on the insulating layer 4. A power supply component 431, a negative ion generator 432 and a negative ion kinetic-energy optimizer 433 are disposed in an outer casing of the negative ion optimization module 43. The negative ion optimization module 43 is provided with a charging connector 434, a switch 435, a negative ion outlet 436, an outer insulating resin plate 437 and an inner conductive metal plate 438 on the outer casing. The negative ion kinetic-energy optimizer 433 has an insulative housing 4331 and a metal vacuum cylinder 4332, and inside the metal vacuum cylinder 4332 has a plurality of metal powders 4333. Wherein the charging connector 434 is electrically connected to the power supply component 431, and the power supply component 431 is electrically connected to the negative ion generator 432 by at least one first power supply line 4311, electrically connected to the negative ion kinetic-energy optimizer 433 by at least one second power supply line 4312, and electrically connected to the inner conductive metal plate 438 by at least one third power supply line 4313. The negative ion generator 432 is electrically connected to the negative ion kinetic-energy optimizer 433 by a conductive negative ion output wire 4321. The negative ion kinetic-energy optimizer 433 has a conductive high-kinetic energy negative ion output wire 4334 electrically connected to the inner conductive metal plate 438. The conductive metal plate 438 is linked with a fullerene release tip to release negative ions that are then released from the negative ion outlet 436 into the human nasal cavity. The insulating layer 4 is partially assembled with the air outlet side 32 by the joint side 41, an air outlet 321 is formed and an air outlet checker 322 is provided between the joint side 41 and a top of the air outlet side 32. Further, the insulating layer 4 has a skin fitted portion 44 in contact with all the portions of the faces, and the skin fitted portion 44 is a silicone material which is capable of effectively adhering to the faces.

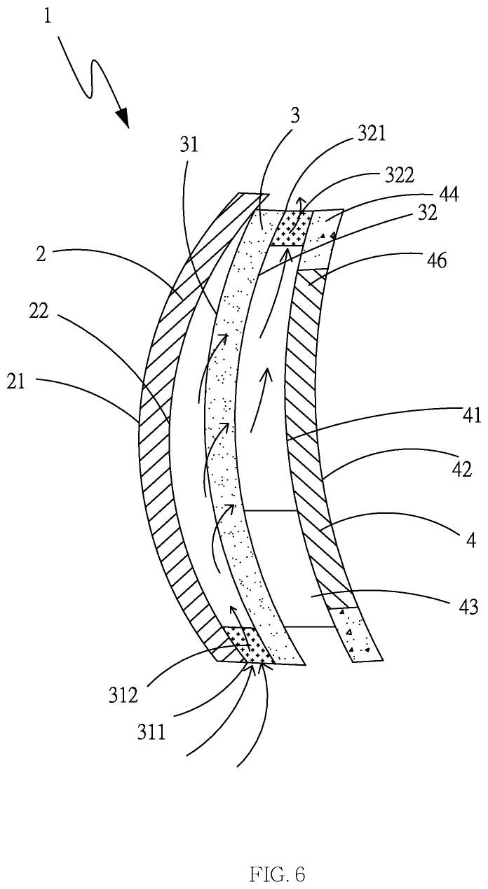

Please refer to the foregoing drawings and FIG. 6 at the same time, FIG. 6 is a first schematic view of the implementation of a preferred embodiment of the negative ion air-permeable mask of the present invention, wherein the negative ion air-permeable mask 1 can be attached to a user's faces by the contact side 42 of the insulating layer 4 when in use, and the skin fitted portion 44 is effectively closely fitted to the skin. Because there is a space formed by downwardly reduction in sizes between the cheeks and the chin of the human faces, that is, a position between the cheekbones and the chin, so that when the insulating layer 4 is attached to the user's faces with the contact side 42, the negative ion optimization module 43 can be accommodated in the space. A side of the negative ion optimization module 43 has a lead-out portion that communicates correspondingly to the nose, and external air enters through the air inlet 311 and passes through the filter layer 3 from the air inlet side 31 to be filtered. The air inlet 311 causes the external air to enter only in single direction with the air intake checker 312, and then is exhausted through the air outlet side 32 and sent out via the air outlet 321, and the air outlet 321 causes the filtered air to be sent out only in single direction with the air outlet checker 322. In addition, the external air is first filtered by the filter layer 3 after passing through the air inlet side 31, and the negative ion optimization module 43 supplies electric power to the negative ion generator 432 and the negative ion kinetic-energy optimizer 433 through the power supply component 431 after the switch 435 is turned on, and the power supply component 431 supplies a high voltage power to the negative ion kinetic-energy optimizer 433 via the second power supply line 4312. After the negative ion generator 432 receives the power of the first power supply line 4311, negative ions generated by the negative ion generator 432 are increased rapidly in quantity through the carbon fiber fullerene release tip connected to the conductive negative ion output wire 4321; the negative ion generator 432 then sends the large incremental negative ions to the negative ion kinetic-energy optimizer 433. The negative ion kinetic-energy optimizer 433 maximizes an amount of the negative ions through the carbon fiber fullerene release tip, and then sends the ions into an environment of a high pressure electrode metal cylindrical vacuum cavity. Electric shock causes the metal powders 4333 to contain kinetic energy, and the negative ions are impacted to become negative ions possessing high kinetic energy. A quantity of high kinetic energy negative ions in the metal vacuum cavity are further increased substantially through the conductive high-kinetic energy negative ion output wire 4334 with the carbon fiber fullerene release tip connected to the inner conductive metal plate 438, and are flushed out from the negative ion outlet 436. The flushed-out high kinetic energy negative ions move quickly to reach the user's nasal cavity as small molecular negative oxygen ions, and this swift movement greatly reduces the chance of being combined by oxygen molecule in the air to form macromolecular negative ions. As a second-tier guarantee other than the initial filtration, the to-be-inhaled air would be purified again by the high kinetic energy negative ions accumulates in a gap between the insulating layer 4 and the faces, allowing the user to directly breathe-in the air rich of small molecular negative ions. The air is circulated sufficiently through a vent between the outer cover layer 2, the filter layer 3 and the insulating layer 4, so that the negative ion air-permeable mask 1 is capable of achieving the efficacies of generating negative ions and having a ventilating effect. In addition, a filter mesh of the filter layer 3 can also be replaced by the user based on different needs to filtrate various different molecules, such as methanol, PM2.5, dust, mites, bacteria, and biochemical toxic gases, thereby it economically facilitates the effect of reusing the negative ion air-permeable mask 1. Furthermore, considering the safety concerns as the negative ion optimization module 43 might have been located very close to the facial skin, the negative ion generator 432 uses a least amount of electricity to supply power in addition to the sealing design of the insulating layer 4. However, because of the need to produce a highest amount of negative ions, the fullerene release tip is used as a release medium in two stages to perform corona discharge. After the negative ion generator 432 generates negative ions, the negative ions pass through a high pressure environment in the sealed metal vacuum cavity, and the negative ions are collided by the metal particles in the negative ion kinetic-energy optimizer 433. The negative ions with high kinetic energy from collision may move fast through the air in a small molecule state and may be quickly inhaled by the human body, which greatly reducing the chance of being combined by water molecules in the air into macromolecular negative ions.

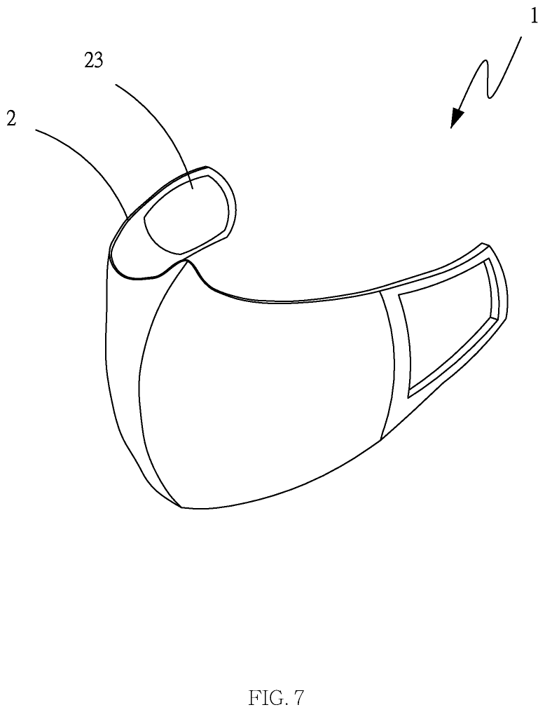

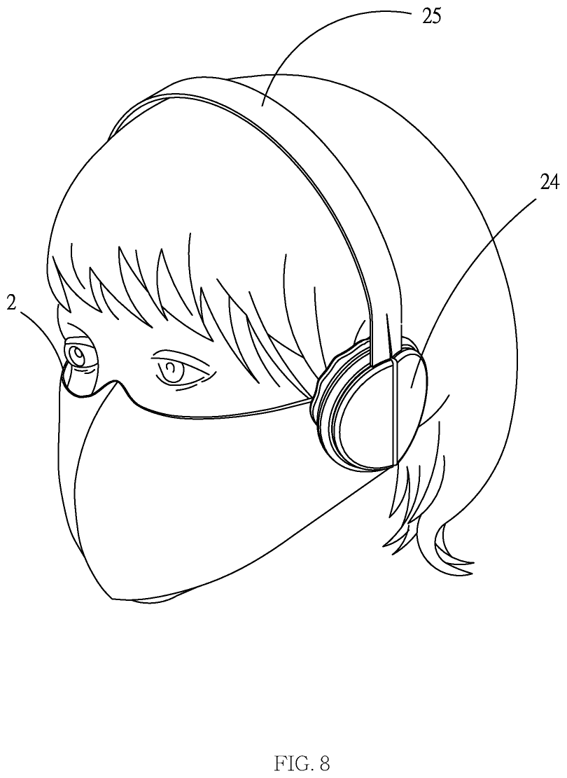

Please refer to FIG. 7 and FIG. 8 simultaneously, which are second schematic view and third schematic view of the implementation of a preferred embodiment of the negative ion air-permeable mask of the present invention. Wherein each side of the outer cover layer 2 has an ear hanger 23, and the ear hangers 23 can be stably sleeved on the ears, so that the user can conveniently wear the negative ion air-permeable mask 1; alternatively, each side of the outer cover layer 2 has an earmuff 24, and a head strip 25 is formed between the two earmuffs 24, the earmuffs 24 can be covered on the ears, and the head strip 25 can be stably worn on the head, so that the user can conveniently wear the negative ion air-permeable mask 1; alternatively the earmuffs 24 can also be an audio effect playing device such as electrically connected headphones, thereby the negative ion air-permeable mask 1 further has the function of headphones in addition to being capable of achieving the efficacies of producing negative ions and having a venting effect.

It is to be understood that the above description is only preferred embodiments of the present invention and is not used to limit the present invention, and changes in accordance with the concepts of the present invention may be made without departing from the spirit of the present invention, for example, the equivalent effects produced by various transformations, variations, modifications and applications made to the configurations or arrangements shall still fall within the scope covered by the appended claims of the present invention.

What is claimed is:

1. A negative ion air-permeable mask, comprising:
an outer cover layer, the outer cover layer having a first side and a second side;
a filter layer, the filter layer being disposed at a position of the second side of the outer cover layer; and
an insulating layer, the insulating layer being disposed at a position of another side of the filter layer opposite to the outer cover layer, the insulating layer having a joint side and a contact side, and the joint side being disposed with a negative ion optimization module.

2. The negative ion air-permeable mask as claimed in claim 1, wherein the filter layer has an air inlet side and an air outlet side, and an air inlet is formed and an air intake checker is provided between the air inlet side and a bottom of the second side.

3. The negative ion air-permeable mask as claimed in claim 2, wherein an air outlet is formed and an air outlet checker is provided between the joint side and a top of the air outlet side.

4. The negative ion air-permeable mask as claimed in claim 1, wherein a power supply component, a negative ion generator and a negative ion kinetic-energy optimizer are disposed in the negative ion optimization module.

5. The negative ion air-permeable mask as claimed in claim 4, wherein the negative ion optimization module is further disposed with a charging connector, a switch, a negative ion outlet, an outer insulating resin plate and an inner conductive metal plate.

6. The negative ion air-permeable mask as claimed in claim 5, wherein the negative ion kinetic-energy optimizer has an insulative housing and a metal vacuum cylinder, and inside the metal vacuum cylinder has a plurality of metal powders.

7. The negative ion air-permeable mask as claimed in claim 6, wherein the charging connector is electrically connected to the power supply component, the power supply component is electrically connected to the negative ion generator by at least one first power supply line, electrically connected to the negative ion kinetic-energy optimizer by at least one second power supply line, and electrically connected to the inner conductive metal plate by at least one third power supply line, the negative ion generator is electrically connected to the negative ion kinetic-energy optimizer via a conductive negative ion output wire, and the negative ion kinetic-energy optimizer further has a conductive high-kinetic energy negative ion output wire electrically connected to the inner conductive metal plate.

8. The negative ion air-permeable mask as claimed in claim 1, wherein the insulating layer has a skin fitted portion.

9. The negative ion air-permeable mask as claimed in claim 1, wherein each side of the outer cover layer has an ear hanger.

10. The negative ion air-permeable mask as claimed in claim 1, wherein each side of the outer cover layer has an earmuff, and a head strip is formed between the two earmuffs.
